(19) United States
(12) Patent Application Publication
(10) Pub. No.: US 2003/0144680 A1
(43) Pub. Date: Jul. 31, 2003

(54) Portable ultrasonic scalpel/cautery device

(75) Inventors: Scott C. Kellogg, Boston, MA (US); Stephen Dimatteo, Warren, RI (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(73) Assignee: Sontra Medical, Inc.

(21) Appl. No.: 10/347,497

(22) Filed: Jan. 21, 2003

Related U.S. Application Data
(60) Provisional application No. 60/349,360, filed on Jan. 22, 2002.

(51) Int. Cl. A61B 17/32

(52) U.S. Cl. 606/169; 606/37

(57) ABSTRACT

A portable ultrasonic scalpel/cautery device is disclosed. According to one embodiment of the present invention, the ultrasonic surgical instrument includes a low voltage battery-powered ultrasonic generator and a handheld ultrasonic surgical instrument. The battery-powered ultrasonic generator generates an ultrasonic signal having a frequency of about 55 kHz.

### CROSS-REFERENCE TO RELATED APPLICATIONS

The present invention claims priority to U.S. Provisional Application No. 60/349,360 filed Jan. 22, 2002, and the contents of which are incorporated herein by reference in their entirety.

### BACKGROUND OF THE INVENTION

1. Field of the Invention

The present invention is directed the field of surgical equipment. Specifically, it is directed to a portable ultrasonic scalpel/cautery device.

2. Description of Related Art

Ultrasonic scalpels have been commercially available from manufacturers such as UltraCision, Inc., and Ethicon Endo-Surgery, Inc. of Cincinnati, Ohio. Examples of related devices are disclosed in U.S. Pat. No. 5,346,502, entitled “Laparoscopic Ultrasonic Surgical Instrument and Methods for Manufacturing the Instruments”; U.S. Pat. No. 5,026,387, entitled “Method and Apparatus for Ultrasonic Surgical Cutting and Hemostatis”; U.S. Pat. No. 6,024,750, entitled “Ultrasonic Curved Blade”; U.S. Pat. No. 6,036,667 and U.S. Pat. No. 6,063,050, both entitled “Ultrasonic Dissection and Coagulation System”; U.S. Pat. No. 6,066,135, entitled “Ultrasonic Operating Apparatus For Vibrating An Ultrasonic Vibrator And Probe Only In A Range Capable Of Constant Current Control And PLL Control And A Control Method For Driving Energy Therefor”; U.S. Pat. No. 5,938,633, entitled “Ultrasonic Surgical Devices”; and U.S. Pat. No. 5,695,510, entitled “Ultrasonic Knife”; U.S. Pat. No. 5,261,922, entitled “Improved Ultrasonic Knife.” The disclosures of each of these patents is incorporated herein by reference in its entirety. The advantages of using power provided at ultrasonic frequencies over other modalities, such as radio frequency (RF) and laser energy are known and well-documented.

Known ultrasonic surgical platforms, however, have several drawbacks. For example, known ultrasonic surgical platforms are generally large and bulky. This is because these devices generally provide a high voltage and a high frequency signal to drive an ultrasonic transducer in a handpiece. For instance, Harmonic Scalpel Generator model GEN01, manufactured by Ethicon Endo-Surgery, is large (approximately 12″×8″×13″) and heavy (approximately 25 lbs.). The generator receives normal AC line voltage (115 VAC @ 60 Hz) and steps this up to a significantly higher voltage (230 VAC @ 60 Hz). The 230 VAC is then rectified, and passed though a silicon controlled rectifier (SCR) switching circuit to produce a DC voltage level that varies between 0 and 250 VDC. This variable voltage ultimately controls the output to the handpiece transducer. The DC voltage is then modulated and filtered to produce the high frequency (55 kHz) signal.

The high voltages used in known generators dictates the large size of the generators. In general, 60 Hz transformers are large and heavy, and, because of the high voltages used, a large amount of insulation and spacing are required. Furthermore, because conventional ultrasonic scalpels and cautery devices draw power from electrical outlets, they require the use of power cords that create a tripping and entanglement hazard in the operating room environment, where the presence of numerous power cords can cause serious safety problems. Still another drawback of conventional outlet-driven devices is that the power line to the universal ground creates an electrical path in communication with other powered devices, increasing leakage current and thus patient risk.

Because of their size, ultrasonic surgical devices are typically a permanent fixture in an operating room. However, doctors who conduct minimal surgical procedures in-office generally are unwilling or unable to incur the expense of purchasing this equipment, and instead use conventional surgical procedures, which may not be as efficient or useful. Furthermore, such devices have no use when power is unavailable, such as in battlefield environments or during temporary power losses.

### SUMMARY OF THE INVENTION

Therefore, a need has arisen for a portable ultrasonic scalpel/cautery device.

It one object of the present invention to provide a portable ultrasonic scalpel/cautery device. It is another object of the present invention that the ultrasonic scalpel/cautery of the present invention provides both cutting and cauterization features.

A portable ultrasonic surgical instrument is disclosed. According to one embodiment of the present invention, the portable ultrasonic surgical instrument includes a low voltage battery-powered ultrasonic signal generator and a handheld ultrasonic surgical instrument operatively connected to the signal generator. The low voltage battery-powered ultrasonic generator generates an ultrasonic signal to power the handheld ultrasonic surgical instrument.

In various embodiments, the device may also include a high frequency step-up transformer, and the handheld ultrasonic surgical instrument may comprise an ultrasonic scalpel, an ultrasonic cautery device or a combined scalpel/cautery device. In one embodiment, the battery-powered ultrasonic signal generator generates an ultrasonic signal having a frequency of about 55 kHz, and in other embodiments, the frequency may be variable.

In still other embodiments, the device may further include a battery, which may be internal or external, and may have a voltage of about 9.6 volts.

### BRIEF DESCRIPTION OF THE DRAWINGS

For a more complete understanding of the present invention, the objects and advantages thereof, reference is now made to the following descriptions taken in connection with the accompanying drawings in which:

FIG. 1 is a drawing of a portable ultrasonic scalpel/cautery device according to one embodiment of the present invention; and,

FIG. 2 is a block diagram schematic of the portable ultrasonic scalpel/cautery device according to another embodiment of the present invention.

### DETAILED DESCRIPTION OF THE EMBODIMENTS

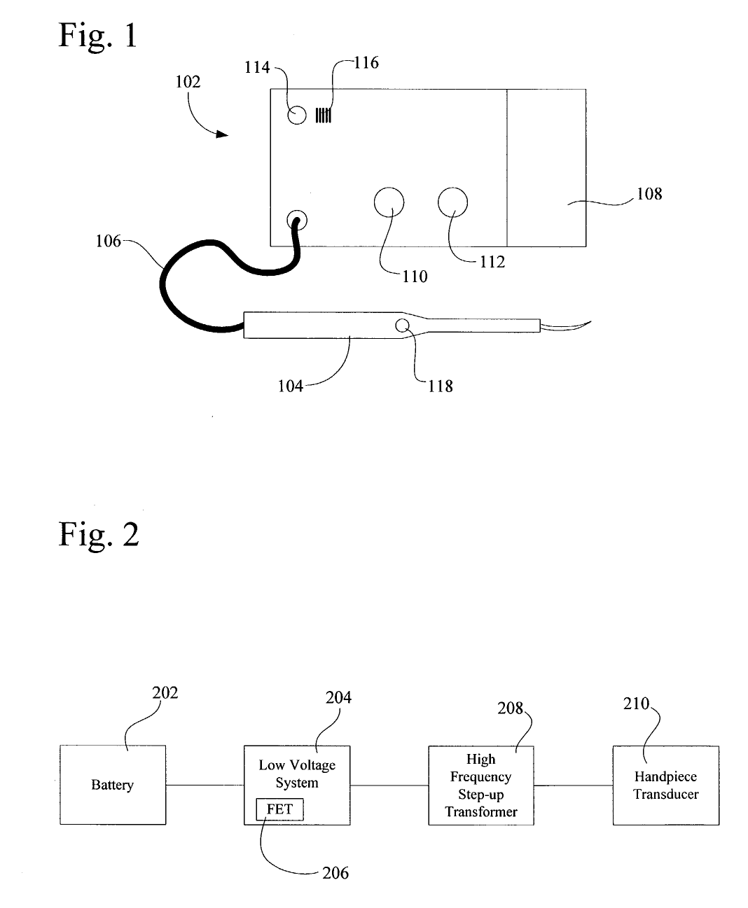

A preferred embodiment of the present invention and its advantages are best understood by referring to FIGS. 1-2 of the drawings.

Referring to FIG. 1, a drawing of a portable ultrasonic scalpel/cautery device according to one embodiment of the present invention is shown. In general, ultrasonic generator 102 generates an ultrasonic signal that drives a transducer (not shown) in handpiece 104, through cable 106. Cable 106 may be permanently affixed to one or both of the ultrasonic generator 102 or the handpiece, or may be connected using removable, and preferably standardized, connectors. Any suitable handpiece, such as those commercially available from U.S. Surgical of Norwalk, Conn., Olympus Optical Co., Ltd. of Tokyo, Japan, and Ethicon Endo-Surgery, Inc. of Cincinnati, Ohio may be used with the present invention.

Ultrasonic generator 102 is a battery-powered, low voltage device. In one embodiment, ultrasonic generator 102 is powered by a relatively small 9.6V battery. The ultrasonic generator 102 uses the battery 108 to produce a variable DC voltage, preferably of between about 0 and 9.6 VDC, and the voltage is modulated to produce the frequency, preferably about 55 kHz, desired to drive the transducer in the handpiece 104. The battery 102 may be stored internally within the device 102, or it may be attached by an external battery-to-generator interface. The device 102 also may be equipped with adaptors to allow use with various different battery types, or with a plug-in unit to provide power to recharge the battery 102. In still other embodiments, the device 102 may be equipped with a battery warning light 114 and/or a speaker 116 to produce an audible low battery warning.

Referring now to FIG. 2, the operation of the portable ultrasonic scalpel/cautery device of the present invention is described in more detail. The device of the present invention comprises a low voltage (less than about 24 volts) battery source 202 that powers a low voltage electrical system 204 that modulates the voltage from the battery to create a desired frequency. The low voltage electrical system 204 may include field effect transistors (FETs) 206 or similar low-voltage circuitry to modulate the battery voltage to the desired final operating frequency. The design and fabrication of such circuitry is well known in the relevant arts. The frequency-modulated output of the low voltage system 204 is then provided to one or more high frequency step-up transformers 208 that convert the low voltage signal to a voltage suitable to drive the handpiece 104. In a preferred embodiment, the voltage may be converted to about 100 V RMS, however other voltages may be used. The output of the high frequency step-up transformer 208 is then supplied to the handpiece transducer 210 to drive the handpiece as either an ultrasonic scalpel or a cautery device.

The present invention takes advantage of the fact that high frequency transformers that operate at ultrasonic frequencies are significantly more efficient than low frequency (i.e. about 60 Hz) transformers; thus, they can be made significantly smaller than low frequency transformers. In one embodiment, ultrasonic generator 102 is approximately five times smaller than the conventional Harmonic Scalpel generator discussed above. In addition, by using a high frequency transformer to step up the voltage after it has been conditioned to the desired frequency, a vast majority of the circuitry in ultrasonic generator 102 can be made using low voltage circuitry. In contrast, conventional generators must condition high voltage line electricity to the desired frequency and amplitude, and therefore a vast majority of the circuitry of known generators is high voltage circuitry. Thus, the size of the signal conditioning circuitry can be significantly reduced using the present invention. For instance, in one embodiment, ultrasonic generator 102 uses miniature and efficient pulse width modulated (PWM) circuitry and Surface Mount Technology (SMT) to minimize its size and power requirements.

In addition, the low voltage circuitry of the present invention can be supplemented, in well-known manners, to easily manipulate the battery voltage to produce various different frequencies and power levels. Various external controls can be added to the device to take advantage of the flexibility of the low voltage circuitry. For example, the control parameters of the handpiece can be readily controlled using an optional frequency control 110 or power control 112. In addition, the handpiece 104 may be equipped with a power switch 118 to turn the device on and off.

It has been found that an ultrasonic generator 102 provided under the trade name SonoPrep by Sontra Medical, Cambridge, Mass. can be combined with a handpiece 104 comprising a model HP052 or HP053, manufactured by Ethicon Endo-Surgery to provide a preferred embodiment of the present invention. The SonoPrep ultrasonic generator operates using a low voltage batter, and provides approximately 20 watts of power, which is similar to the power output of conventional generators set at the lower power setting, and is sufficient to power the handpiece for a useful operation life before requiring a battery change or recharge.

The present invention provides several advantages. The size of the device of the present invention permits its use in doctors' offices, which may reduce the need for hospitalization for certain procedures. Further, the device of the present invention provides cauterization, which increases the efficiency of surgical procedures. The present invention also may be used in environments where power is not otherwise available, such as battlefields and roadsides, and is small and light enough to fit into a personal carrying device, such as a backpack, to allow easy portability by field medics and emergency medical personnel.

### EXAMPLE

In order to better understand the present invention, an example of a portable ultrasonic scalpel/cautery device according to one embodiment is provided. This examples does not limit the present invention in any way, and is intended to illustrate an embodiment and potential application of the present invention. In the exemplary example, the ultrasonic generator is a SonoPrep ultrasonic generator, available from Sontra Medical, Cambridge, Mass. The SonoPrep uses a 9.6 V battery, and provides an output voltage that varies between 10 and 80 VRMS at 55 kHz. The SonoPrep device is about the size of a paperback novel, and is relatively small compared to conventional ultrasonic signal generators. The handpiece was manufactured by UltraCision, Inc. (which became part of Ethicon Endo-Surgery, Inc.), and is functionally equivalent to a model HP052 or HP053 handpiece, manufactured by Ethicon Endo-Surgery.

In order to test the device, the described ultrasonic scalpel/cautery device was brought in contact with chamois, which it was able to cut and char. This is a standard test that shows initial efficacy for use as a scalpel and cautery device.

For purposes of clarity of understanding, the foregoing invention has been described in some detail by way of illustration and example in conjunction with specific embodiments, although other aspects, advantages and modifications will be apparent to those skilled in the art to which the invention pertains. The foregoing description and examples are intended to illustrate, but not limit the scope of the invention. Modifications of the above-described modes for carrying out the invention will be apparent to persons of skill in medicine, bacteriology, infectious diseases, pharmacology, and/or related fields, and all modifications are intended to be within the scope of the invention, which is limited only by the appended claims.

All publications and patent applications mentioned in this specification are indicative of the level of skill of those skilled in the art to which this invention pertains. All publications and patent applications mentioned herein are incorporated by reference to the same extent as if each individual publication or patent application was specifically and individually indicated to be incorporated by reference.

We claim:

1. A portable ultrasonic surgical instrument, comprising:
a low voltage battery-powered ultrasonic generator; and
a handheld ultrasonic surgical instrument operatively connected to the low voltage battery-powered ultrasonic generator;
wherein the low voltage battery-powered ultrasonic generator generates an ultrasonic signal to power the handheld ultrasonic surgical instrument.

2. The portable ultrasonic surgical instrument of claim 1, further comprising a high frequency step-up transformer.

3. The portable ultrasonic surgical instrument of claim 1, wherein the handheld ultrasonic surgical instrument comprises an ultrasonic scalpel.

4. The portable ultrasonic surgical instrument of claim 1, wherein the handheld ultrasonic surgical instrument comprises an ultrasonic cautery device.

5. The portable ultrasonic surgical instrument of claim 1, wherein the handheld ultrasonic surgical instrument comprises an ultrasonic scalpel and a cautery device.

6. The portable ultrasonic surgical instrument of claim 1, wherein the ultrasonic signal has a frequency of about 55 kHz.

7. The portable ultrasonic surgical instrument of claim 1, wherein the ultrasonic signal is adjustable.

8. The portable ultrasonic surgical instrument of claim 1, further comprising a battery.

9. The portable ultrasonic surgical instrument of claim 8, wherein the battery is an internal battery.

10. The portable ultrasonic surgical instrument of claim 8, wherein the battery is an external battery.

11. The portable ultrasonic surgical instrument of claim 8, wherein the battery has a battery voltage of about 9.6 volts.

12. The portable ultrasonic surgical instrument of claim 11, further comprising a high frequency step-up transformer adapted to increase the battery voltage to about 80 volts.
